(19) United States
(12) Patent Application Publication
(10) Pub. No.: US 2021/0005293 A1
(43) Pub. Date: Jan. 7, 2021

(54) SYSTEM AND METHOD FOR PROVIDING ACCESS OF A USER'S HEALTH INFORMATION TO THIRD PARTIES

(71) Applicant: Patientory, Inc., Atlanta, GA (US)

(72) Inventor: Chrissa Tanelia McFarlane, Atlanta, GA (US)

(21) Appl. No.: 16/584,573

(22) Filed: Sep. 26, 2019

Related U.S. Application Data
(60) Provisional application No. 62/737,011, filed on Sep. 26, 2018.

(51) Int. Cl. G16H 10/60; G16H 80/00; H04L 9/06

(52) U.S. Cl. CPC G16H 10/60; H04L 9/0637; G16H 80/00

(57) ABSTRACT

A system and a method for providing access of a user's health information are described. The method comprises providing a Health Information Exchange (HIE) server implemented over a blockchain network. The HIE server stores health information of a plurality of users. Further, a user device is present in communication with the HIE server. An access of the user's health information is provided to a third party, based on a user's permission, for performing a research on the user's health information. The user's health information is stored on the blockchain network. The user may be allowed to access results of the research stored on the blockchain network.

### Other Related Applications

The present application is a U.S. Non-Provisional Patent Application claiming priority of U.S. Provisional Patent Application Ser. No. 62/737,011 filed on Sep. 26, 2018, which is hereby incorporated by reference.

### BACKGROUND OF THE INVENTION

### Field of the Invention

The present disclosure is generally related to a health care network, particularly a health care network implemented over blockchain, and more particularly related to a method for providing access of a user's health information to third parties.

### Description of the Related Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner. The blocks can be added to the blockchain in a linear and chronological order. The blockchain helps to store and track data in a secured manner. Currently, the blockchain is used in various fields such as gaming and gambling, diamond industry, real estate, medical industry, or e-voting.

Further, blockchain can be utilized in various fields involving multi-parties for accessing patient information when authorized to conduct studies on various healthcare and medical aspects. These studies can be conducted, for example, for new surgical procedures, new treatments, a disease diagnosis, or a pharmaceutical drug. In such a scenario, multi-parties will have access to specific patient's medical records through blockchain, which can be updated through users, or third parties.

However, it becomes tedious to maintain a high security when the updated information is accessed by multiple parties through various devices. Further, such process results in increasing overhead cost of conducting research studies. Therefore, there is a need for an improved system that may perform studies on large data sets without infringing on privacy concerns, and without affecting overhead costs of conducting the research studies.

Applicant believes that a related reference corresponds to U.S. patent No. 2019/0213333 for a Decentralized Data Authentication System for Creation of Integrated Lifetime Health Records. Applicant believes another related reference corresponds to U.S. patent No. 2018/0060496 for a Blockchain-Based Mechanisms for Secure Health Information Resource Exchange. None of these references, however, teach of a system that gathers and collects data to be analyzed for conducting research studies in a manner that is quick, efficient and highly secure.

### SUMMARY OF THE INVENTION

It is one of the objects of the present invention to provide a system and method for providing access of a user's health information to third parties.

It is another object of the present invention to provide a system and method for providing access of a user's health information to third parties via a blockchain.

It is still another object of the present invention to provide a system and method for providing access of a user's health information to third parties that is highly secure.

It is another object of the present invention to provide a system and method for providing access of a user's health information to third parties for conducting research.

It is still yet another object of the present invention to provide a system and method for providing access of a user's health information to third parties that reduces overhead costs of third parties conducting research.

It is another object of the present invention to provide a system and method for providing access of a user's health information to third parties that can gather and manage data in an efficient manner.

It is still yet another object of the present invention to provide a system and method for providing access of a user's health information to third parties that aids in making medical advances.

It is an object of the present invention to provide a system and method for providing access of a user's health information to third parties that is inexpensive to implement and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a network connection diagram of a Health Information Exchange (HIE) system for providing access to user's health information, according to various embodiments.

FIG. 2 illustrates a method for symmetric encryption of data, according to various embodiments.

FIG. 2A illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates a system for storing and accessing data in the health care network implemented for example over a blockchain network, according to various embodiments.

FIG. 6 illustrates exemplary information stored in a research database, according to various embodiments.

FIG. 6A illustrates exemplary information stored in an authorization database, according to various embodiments.

FIG. 6B illustrates exemplary information stored in a research results database, according to various embodiments.

FIG. 7 illustrates a flowchart showing a method performed by a research access module, according to various embodiments.

FIG. 8 illustrates a flowchart showing a method performed by a research module, according to various embodiments.

FIG. 9 illustrates a flowchart showing a method performed by a research upload module, according to various embodiments.

### DETAILED DESCRIPTION OF THE EMBODIMENTS OF THE INVENTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of various embodiments of the present disclosure, various embodiments of systems and methods will be described.

Embodiments of the present disclosure will be described more fully hereinafter with reference to the accompanying drawings in which like numerals may represent like elements throughout the several figures, and in which various example embodiments are shown. Various embodiments may, however, be embodied in many different forms and should not be construed as limited to the embodiments set forth herein. The examples set forth herein are non-limiting examples and are merely examples among other possible examples.

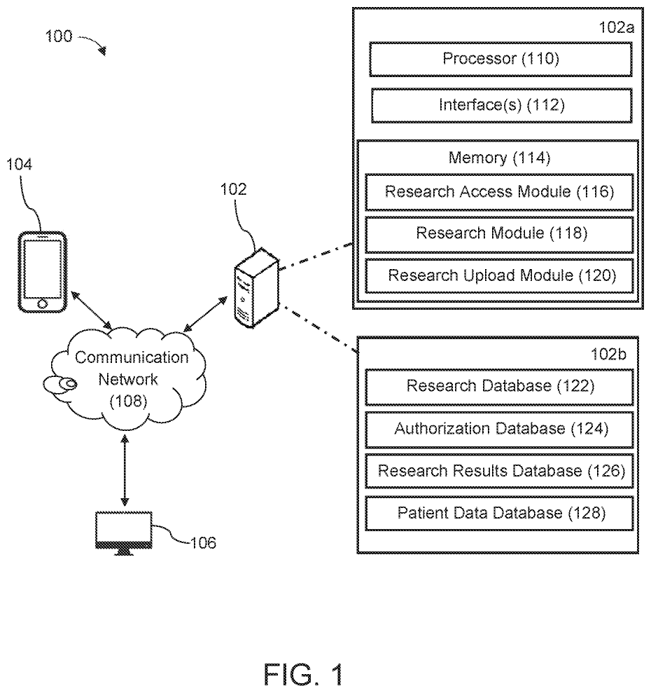

FIG. 1 illustrates a network connection diagram 100 of a Health Information Exchange (HIE) system 102 for providing access to user's health information. The HIE server 102 may comprise one or more user interfaces. The one or more user interfaces may be accessed by one or more user via one or more user devices 104. The HIE server 102 may be connected with a user device 104 and a third party device 106, through a communication network 108.

The communication network 108 may be a wired and/or a wireless network. The communication network 108, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE server 102 may comprise a group of components 102a required for providing the access to the user's health information. The group of components 102a may include a processor 110, interface(s) 112, and a memory 114. The memory 114 may comprise modules implemented as a program. In various embodiments, the memory 114 may comprise a research access module 116, a research module 118, and a research upload module 120. Further, the HIE server 102 may comprise or may be connected with a group of databases 102b which may include a research database 122, an authorization database 124, research results database 126, and a patient data database 128.

The processor 110 may execute an algorithm stored in the memory 114 for providing the access to the user's health information. The processor 110 may also be configured to decode and execute any instructions received from one or more other electronic devices or server(s). The processor 110 may include one or more general purpose processors (e.g., microprocessors) and/or one or more special purpose processors (e.g., digital signal processors (DSPs) System On Chips (SOCs) Field Programmable Gate Arrays (FPGAs), or Application-Specific Integrated Circuits (ASICs)). The processor 110 may be configured to execute one or more computer-readable program instructions, such as program instructions to carry out any of the functions described in this description.

The interface(s) 112 may help an operator to interact with the HIE server 102. The interface(s) 112 may either accept inputs from users or provide outputs to the users or may perform both the actions. In various embodiments, a user can interact with the interface(s) 112 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or any combination of the above. Further, the interface(s) 112 may be implemented as a Command Line Interface (CLI), a Graphical User Interface (GUI), a voice interface, or a web-based user-interface.

The memory 114 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. The memory 114 may comprise modules implemented as a program. In various embodiments, the memory 114 may comprise the research access module 116, the research module 118, and the research upload module 120.

In various embodiments, several users may interact with the HIE server 102, using a user device 104. Although a single user device has been illustrated, several user devices could similarly be connected to the communication network 108. Further, each of the user devices may have a device ID. In various embodiments, the device ID may be a unique identification code such as an International Mobile Equipment Identity (IMEI) code or a product serial number. It should be noted that a user may use a single user device or multiple user devices. Further, multiple users may use a single user device or multiple user devices. Further, the one or more users may receive and/or provide healthcare related products and services. The one or more users may include patients, for example but not limited to, family and friends of the patients, hospitals, physicians, nurses, specialists, pharmacies, medical laboratories, testing centers, insurance companies, or Emergency Medical Technician (EMT) services.

The user device 104 may be a stationary device, a portable device, or a device accessed remotely. The user device 104 may be, but not limited to, a computer, a laptop, a tablet, a mobile phone, a smartphone, an Internet of Things (IoT) device, or a smart watch. In various embodiments, the user device 104 may include an imaging device that may be configured to capture a visual graphical element. The visual graphical element such as, but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 104. In various embodiments, the hardware camera sensor may be embedded in the user device 104. In various embodiments, the imaging device may be located external to the user device 104. In various embodiments, the imaging device may be connected to the user device 104 wirelessly or via a cable. It should be noted that image data of the visual graphical element may be transmitted to the user device 104 via the communication network 108.

In various embodiments, the imaging device may be controlled by applications and/or software(s) configured to scan a visual graphical code. In various embodiments, a camera may be configured to scan a QR code. Further, the applications and/or software(s) may be configured to activate the camera present in the user device 104 to scan the QR code. In various embodiments, the camera may be controlled by a processor natively embedded in the user device 104. In various embodiments, the imaging device may include a screen capturing software (for example, screenshot) that may be configured to capture and/or scan the QR code on a screen of the user device 104.

In various embodiments, the user device 104 may collect information related to the user's daily health status. The information may include monitoring, for example, heart rate, blood pressure, or steps per day. In various embodiments, the information may be stored in the patient data database 128. It should be noted that the patient data database 128 may be populated by the third parties such as an individual belonging to, for example, hospitals, insurance companies, Contract Research Organizations (CROs), and drug companies. The information may include, for example, new prescriptions, undergone procedures, or diagnosed diseases.

Further, the user device 104 may be used to accept and enter into smart contracts that may allow access to the user's health information stored on a blockchain network. The information may be accessed by the third party. It should be noted that the blockchain network may refer to be an independent entity that owns and operates blockchain for use by others, which may enhance security. In various embodiments, a blockchain may be a continuously growing list of records, which can be referred to as blocks, which may be linked and secured using cryptography. Each block may contain a hash (e.g., private or public keys needed for block chain access) of a previous block, a timestamp, and transaction data, for which the transaction data may be health records and data. Further, each block may be inherently resistant to modification of the data due to, for example, management by a peer-to-peer network adhering to a protocol for internode communication and validating new blocks.

In various embodiments, the group of databases 102b may be connected to the HIE server 102. In various embodiments, the group of databases 102b may be implemented over the blockchain network (such as a PTOYNet blockchain network or a PTOYNet Ethereum™ Blockchain network) and may be present as different databases installed at different locations. The group of databases 102b may include the research database 122, the authorization database 124, the research results database 126, and the patient data database 128. The group of databases 102b may be configured to store data belonging to different users and data required for functioning of the HIE server 102. Different databases may be used in accordance with various embodiments; however, a single database may also be used for storing the data. Usage of the different databases may also allow segregated storage of different data and may thus reduce time to access desired data. In various embodiments, the data may be encrypted, time-dependent, piece-wise, and may be present as subsets of data belonging to each user. In various embodiments, the data may represent the results of one medical test in a series of multiple medical tests.

In various embodiments, the group of databases 102b may operate collectively or individually. Further, the group of databases 102b may store data as tables, objects. or other structures. Further, the group of databases 102b may be configured to store data required or processed by the HIE server 102. The data may include, but not limited to, a patient medical history, medical charts, medications, prescriptions, immunizations, test results, allergies, insurance provider, or billing information. Further, the data may be time-dependent and piece-wise. Further, the data may represent a subset of data for each patient. In various embodiments, the data may represent results of a medical test in a series of multiple medical tests. Further, the data may be securely stored. In various embodiments, the data may be encrypted.

In various embodiments, information stored in the group of databases 102b may be accessed based on users' identities and/or the users' authorities. The users' identities may be verified in one or more ways such as, but not limited to, biometric authentication or bio-authentication, password or PIN information, user device registrations, a second-level authentication, or a third-level authentication. In various embodiments, the users' identities may be verified by the HIE server 102. Information provided by the users in a real-time may be used, by the HIE server 102, to confirm the users' identities. In various embodiments, the users' identities may be verified using a name, a password, one or security questions, or a combination thereof. In various embodiments, a user may be identified using an encryption key and/or a decryption key.

In various embodiments, the data stored in the group of databases 102b may be accessed at different levels, for example using a first level subsystem and a second level subsystem. In various embodiments, a user may directly access the first level subsystem. To access data stored in the second level subsystem, the second level subsystem may need to be accessed through the first level subsystem. It should be noted that the communication between the first level subsystem and the second level sub-system may be encrypted. In various embodiments, the second level subsystem may be implemented over a blockchain network (such as a PTOYNet blockchain network). In various embodiments, the PTOYNet blockchain network may be used to implement smart contracts.

In various embodiments, a primary care physician may input data into the HIE server 102 using the user device 104. The data may be processed by the first level subsystem and the second level subsystem. The data may be stored on the first level subsystem and/or the second level subsystem of the HIE server 102. The data may include, but not limited to, one or more instructions to a patient to see a physician specialist. Further, the data may be stored in one or more blockchains of the second level subsystem. The patient may be able to access the data relating to the patient's care provided by the primary care physician. The patient may be able to retrieve the data using the user device 104 of the patient.

In accordance with various embodiments, the patient may communicate with the physician specialist using the HIE server 102. It should be noted that the physician specialist may be able to access the data of the patient from the first level subsystem and/or the second level subsystem. Further, the physician specialist may be able to communicate with the patient. It should be noted that some, all, or substantially all communications between the primary care physician, the physician specialist and the patient may be stored and may be accessible on a blockchain network.

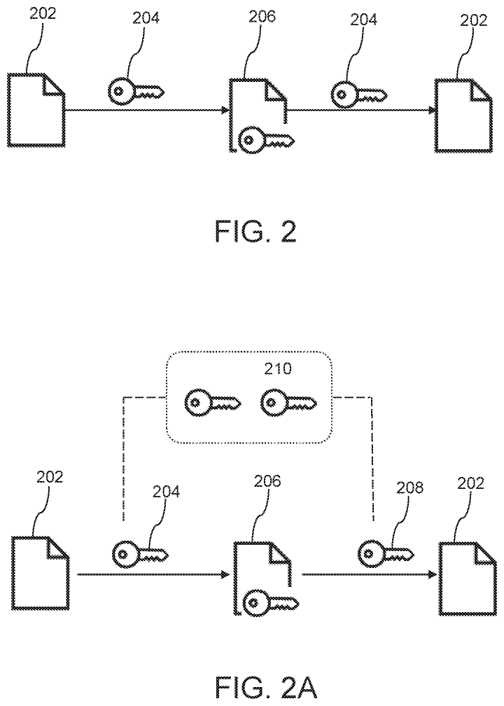

FIG. 2 illustrates a method for symmetric encryption of data, according to various embodiments. Original data 202 may be encrypted using a key 204 to obtain an encrypted data 206. The encrypted data 206 may be decrypted using the key 204 to obtain back the original data 202. It should be noted that encryption and decryption of the data may be performed using a same key. Further, one or more parties involved in a communication may have the same key to encrypt and decrypt the data.

FIG. 2A illustrates a method for asymmetric encryption of data, according to various embodiments. Original data 202 may be encrypted using a key 204 to obtain encrypted data 206. The encrypted data 206 may be decrypted using another key 208 to obtain the original data 202. It should be noted that encryption and decryption of the data may be performed using different keys i.e. a key pair 210.

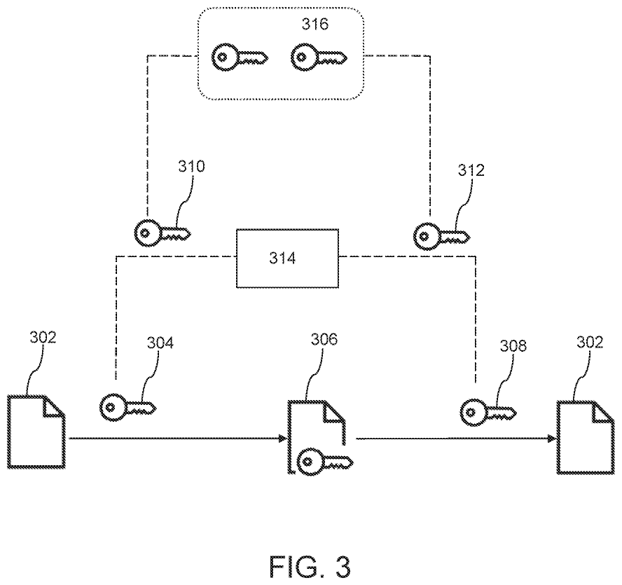

FIG. 3 illustrates a method for hybrid encryption of data, according to various embodiments. Both symmetric encryption and asymmetric encryption techniques may be used in tandem. In various embodiments, the symmetric encryption technique may be used to encrypt data 302 using a symmetric key 304 for producing encrypted data 306. The encrypted data 306 may be decrypted using another symmetric key 308 for obtaining data 302 (or back data). Further, a public key 310 may be used to encrypt the symmetric key 304 and a private key 312 may be used to encrypt the symmetric key 308, stored as an encrypted key 314. The public key 310 and the private key 312 may for a key pair 316.

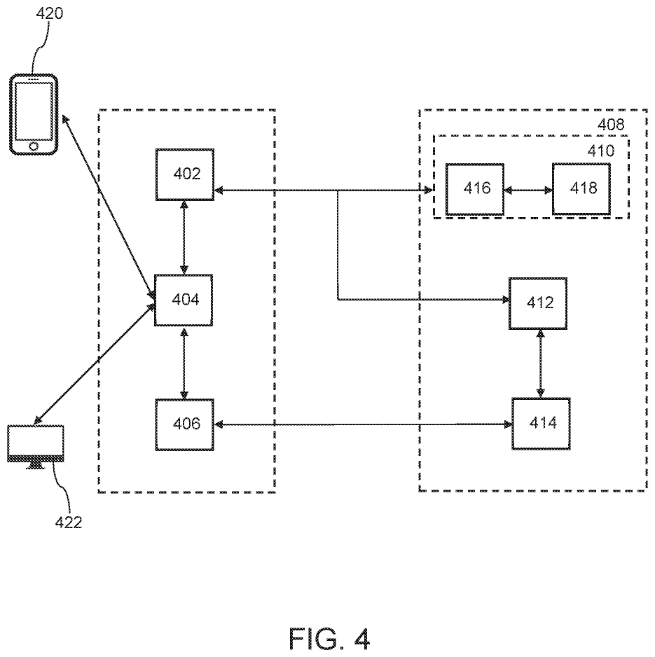

In accordance with various embodiments, FIG. 4 illustrates a system for storing and accessing data in a health care network, the first level subsystem may include a core service component 402 and a Remote Procedure Call (RPC) component 404. In various embodiments, the second level subsystem may include a blockchain node component 406 (e.g., quorum blockchain node component 406). In various embodiments, the first level subsystem may include the core service component 402, and the second level subsystem may include the RPC component 404 and the quorum blockchain node component 406. Further, the core service component 402 of the first level subsystem may be present in communication with third-party servers and databases of a hospital computing network 408. The hospital computing network 408 may include an Interplanetary File System (IPFS) module 410, an EHR synchronization service 412, and a blockchain node 414 (e.g., quorum blockchain node 414). Further, the IPFS module 410 may include an IPFS manager 416 and an IPFS node 418. The quorum blockchain node component 406 of the second level subsystem may communicate with the quorum blockchain node 414 of the hospital computing network 408. Patients may access the health care network for storing data through a user device 420, and a representative of a hospital may access the health care network through another user device 422.

In accordance with various embodiments, the representative of the hospital may want to synchronize Electronic Health Record (EHR) data of a patient. The first level subsystem and the second level subsystem may ask the patient for permission to allow a representative of the hospital to store the EHR data of the patient, through the IPFS module 410. Based at least on the permission granted by the patient, a signed transaction may be created to confirm the permission of the hospital to store the EHR data. Further, the signed transaction may activate a smart contract that may add hospital identification information such as a blockchain address to a list of permitted users.

In accordance with various embodiments, the signed transaction may be transmitted from the user device to the RPC component 404 of the first level subsystem and/or the second level subsystem. The RPC component 404 may communicate the signed transaction to the quorum blockchain node component 406 of the second level subsystem. The quorum blockchain node component 406 may activate one or more smart contracts. Thereafter, the quorum blockchain node component 406 may revise a state of one or more blockchains.

In accordance with various embodiments, based at least on the permission granted by the patient, the EHR synchronization service may obtain a list of patients from the RPC component 404. Further, the EHR synchronization service may confirm whether the patient has granted permission. Based at least on the permission, the first level subsystem and the second level subsystem may obtain the EHR data and may calculate a hash function for the EHR data. The HIE server 102 may match the hash function of the EHR data with a hash function for the patient blockchain on the quorum blockchain node component 406 of the second level subsystem. If the hash function of the EHR data matches with the hash function for the patient blockchain on the quorum blockchain node component 406 of the second level subsystem, the EHR data of the patient may remain unchanged.

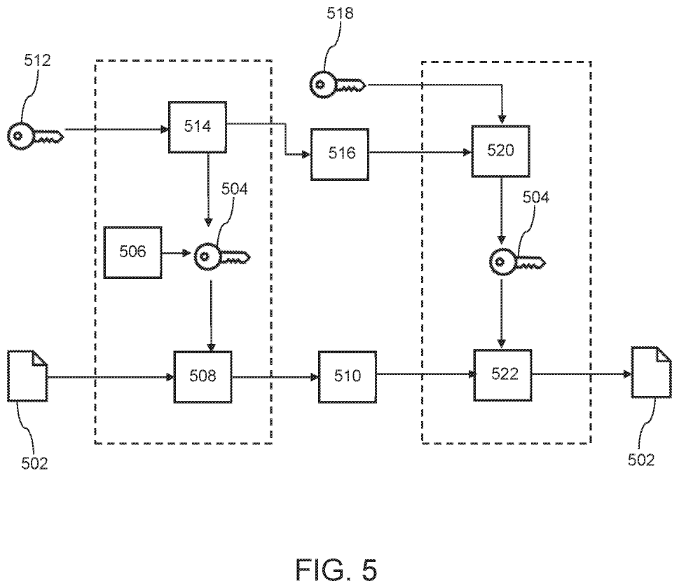

In accordance with various embodiments, FIG. 5 illustrates a system for storing and accessing data in a health care network implemented specifically over a blockchain network (such as a PTOYNet blockchain network or a PTOYNet Ethereum™ blockchain network), the HIE server 102 may execute an application for determining permission from the user for obtaining EHR data 502. In various embodiments, if the user grants the permission, the HIE server 102 may obtain the EHR data 502 for calculating a hash function for the EHR data 502. Further, the HIE server 102 may match the hash function of the EHR data 502 with a hash function for the user blockchain on the quorum blockchain node of the second level sub-system. In various embodiments, if the two hash matches, there is no change to the user's EHR data 502. In various embodiments, the two hash functions do not match, the HIE server 102 may generate a random string (i.e. secret key 504), through a random key generator 506. The secret key 504 may be used for Advanced Encryption Standard (AES) encryption of the EHR data 502, in an AES encryptor 508, for generating encrypted EHR data 510.

In accordance with various embodiments, the key 504 may then be encrypted by, for example, a Rivest-Shamir-Adleman (RSA) public key 512 of the patient, in an RSA encryptor 514, to generate an encrypted secret key 516. The HIE server 102 may also send the encrypted EHR data 510 to the core service component 402 for forwarding the data to the IPFS manager 416 of the hospital computing network 408 for storage. The IPFS manager 416 may send an IPFS hash function to the core service component 402 for further sending the IPFS hash function to EHR synchronization service 412. The EHR synchronization service 412 may further update the patient smart contract with the new IPFS hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to a RPC component 404 for granting permission to the hospital representative to view the EHR data 502. Once the permission is granted, the signed transaction may be added to the quorum blockchain node 414 and a new smart contract will be created for a blockchain corresponding to the hospital representative. After adding the signed transaction, the hospital representative may be able to view the EHR data 502 of the user, on a device.

In various embodiments, in order to view the EHR data 502 on the device, the HIE server 102 may collect the encrypted EHR data 510 from the user's blockchain and may decrypt the encrypted EHR data 510 using patient's RSA private key 518. The HIE server 102 may decrypt the encrypted secret key 516, in an RSA decryptor 520, using RSA private key of the hospital representative. The encrypted EHR data 510 may be decrypted using the RSA public key 512 of the hospital representative, in an AES decryptor 522. Further, the HIE server 102 may load the decrypted EHR data 502 to the smart contract previously created for the hospital representative.

After loading the decrypted EHR data 502, the RPC component 404 may obtain the signed transaction from the patient's user device and transmit the signed transaction to the quorum blockchain node component 406 of the second level subsystem. The quorum blockchain node component 406 may confirm ownership of the signed transaction and may execute the smart contract for the hospital representative to view the user's data.

In various embodiments, the patient may decline permission for the hospital representative to have access to the EHR data 502. For example, the user through a user device may send a signed transaction revoking permission to the RPC component 404. The RPC component 404 may forward the signed transaction to the quorum blockchain node component 406 of the second level subsystem. The quorum blockchain node component 406 may confirm ownership of the signed transaction and may delete the smart contract previously created to allow the hospital representative to have access to the patient's EHR data 502.

In various embodiments, the HIE server 102 may comprise a health record network for an intermediary enabling sharing of user's medical records with providers. For enabling sharing, the user may grant specific permissions to the providers for accessing parts of the user's medical records stored in the patient data database 128 implemented over the blockchain network. The user may also grant specific permissions to modify the user's medical records in the patient data database 128. In various embodiments, the user may comprise of any users constituting a value chain, such as doctors, nurses etc. In various embodiments, the user may be remote doctors logging into the HIE server 102 or doctors present in hospitals.

In various embodiments, the HIE server 102 may communicate with the third party device 106, through the communication network 108. The third party device 106 may be operated by the third party. The third party may be an individual belonging to, for example, one of hospitals, insurance companies, Contract Research Organizations (CROs), and drug companies. Further, the third party device 106 may include an interface i.e., Graphical User Interface (GUI).

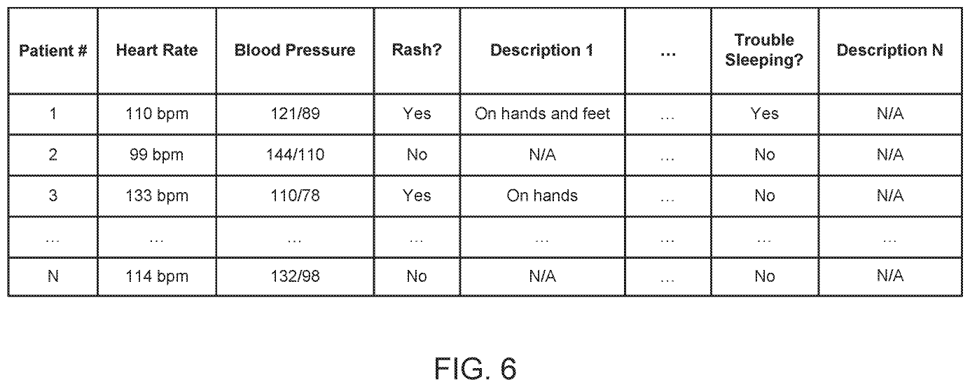

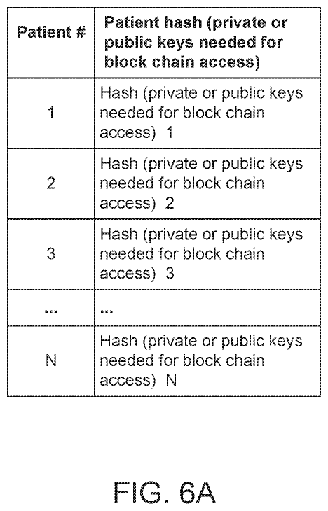

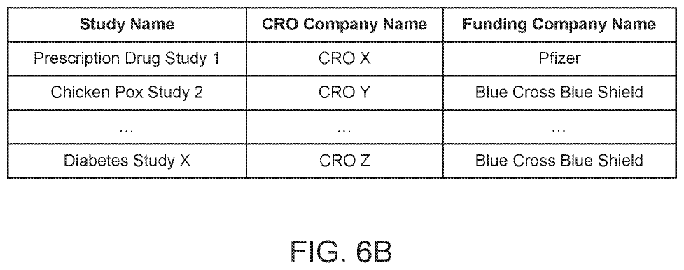

In various embodiments, the research database 122 may be configured to store information related to a study of interest that the third party is conducting. The third party may be an individual belonging individual belonging to, for example, one of hospitals, insurance companies, Contract Research Organizations (CROs), and drug companies. In various embodiments, the CROs may provide pharmaceutical, biotechnology, and medical device industries research in the form of research services outsourced on a contract basis. Further, the information stored may vary based on a type of study being conducted. The study may be conducted on a new prescription drug after an open heart surgery, a new surgery procedure, a new treatment for a disease, or a disease itself. As shown in FIG. 6, for example, the research database 122 may store information about the patients who had experienced, for example, certain side effects, a rate of recovery, and basic vital signs. In various embodiments, the CROs may use such information for research studies.

In various embodiments, the authorization database 124 may be configured to store information about what user's health information may be used by the third party for the research studies. Further, the authorization database 124 may store a unique hash (e.g., private or public keys needed for block chain access) for authorized users and the third parties. It should be noted that the user may use the information for entering into a smart contract post through the user device 104. As shown in FIG. 6A, for example, the authorization database 124 may store the unique hash function for the users that authorized the use of the user's health information. It should be noted that the authorization database 124 may be populated by the smart contracts in place between the organization that hired the CROs and the users (i.e., patients). It should be noted that each patient has a unique hash (e.g., private or public keys needed for block chain access), so when information is updated on the user device 104 or through an entity on the third party device 106, the blockchain associated with the user, which is identified through the unique hash (e.g., private or public keys needed for block chain access), may be updated.

In various embodiments, the research results database 126 may be configured to store information related to results of the research studies. In various embodiments, results of the research studies conducted by the CROs, may be accessed by the user whose medical data is used for the research study. It should be noted that the research results database 126 may be populated by the research upload module 120. As shown in FIG. 6B, for example, the research results database 126 may store information such as study name, CRO company name, and funding company name. For example, the research results database 126 may store information about CROs studies and research that are using the user's health information. Further, the research results database 126 may be accessed by the research access module 116 to help the user to access the results of the research studies conducted by the CROs on the blockchain network.

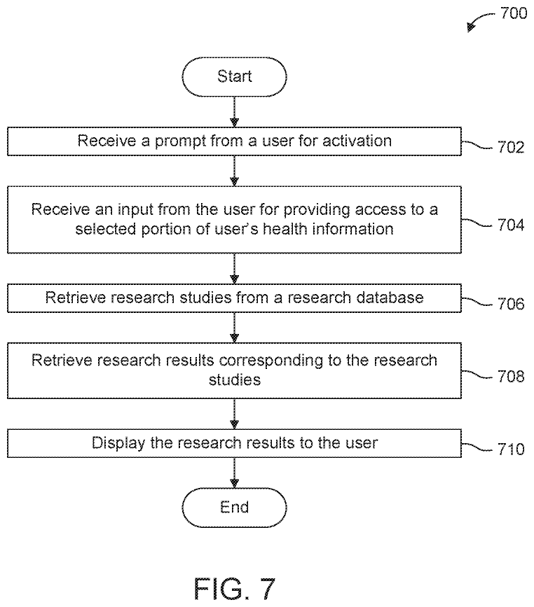

FIG. 7 illustrates a flowchart 700 showing an example method performed by the research access module 116, according to various embodiments. An example process utilizing the research access module 116 will now be explained with reference to, for example, the flowchart 700 shown in FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The research access module 116 may receive a prompt from a user for activation, at step 702. The research access module 116 may receive an input from the user for providing access to a selected portion of the user's heath information, to the third party, at step 704. In various embodiments, the third party may be CROs. It should be noted that the user may get payments or coupon for future drugs, for providing access to the user's health information. Further, the research access module 116 may provide keys (e.g., public and private keys) to the user for allowing access of the user's health information stored over the blockchain network, to others. It should be noted that the access may be granular to very specific types of data. Further, the keys may be stored in the authorization database 124.

The research access module 116 may retrieve research studies from the research database 122, at step 706. In various embodiments, the research studies may be retrieved if the user's health information is allowed by the user to be shared with the CROs. The research access module 116 may retrieve research results corresponding to the research studies, at step 708. The research results may be retrieved from the research results database 126. Thereafter, the research access module 116 may display the research results to the user for review, at step 710.

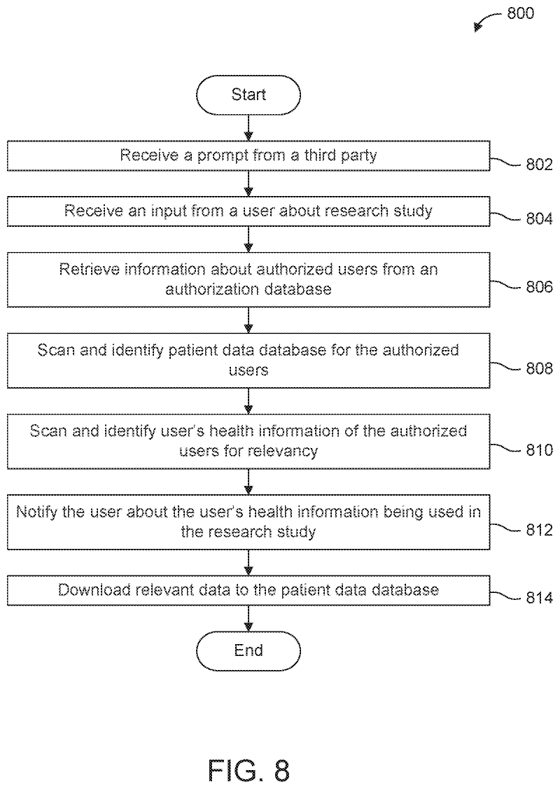

FIG. 8 illustrates a flowchart 800 showing an example method performed by the research module 118, according to various embodiments. An example process utilizing the research module 118 will now be explained with reference to, for example, the flowchart 800 shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The research module 118 may receive a prompt from the third party, at step 802. In various embodiments, the third party may be CROs. The research module 118 may receive an input from the user about the research study, at step 804. In various embodiments, the input information may correspond to tracking side effects of a new prescription drug. The research module 118 may retrieve information about authorized users (i.e., authorized patients) from the authorization database 124, at step 806. In various embodiments, the information retrieved from the user may be analyzed for research. It should be noted that the information may include hash keys (e.g., private or public keys) from the authorized users.

Returning to flowchart 800 illustrated in FIG. 8, the research module 118 may scan and identify the patient data database 128 for the authorized users, at step 808. In various embodiments, the research module 118 may scan encrypted patient data database 128 for the data of the authorized users by corresponding the hash (e.g., private or public keys needed for block chain access) stored in the authorized database 124 to all hash (private or public keys needed for block chain access) stored in the patient data database 128. The research module 118 may scan and identify the user's health information of the authorized users for relevancy, at step 810. For example, for a new prescription drug, the research module 118 may scan all data to look for the users taking that drug.

Returning to flowchart 800 illustrated in FIG. 8, the research module 118 may notify the user about the user's heath information being used in the research study, at step 812. In various embodiments, the research module 118 may notify the user by storing a copy of basic research information on the research database 122. The basic research information may be used by the user in the future. Thereafter, the research module 118 may download relevant data to the patient data database 128, at step 814.

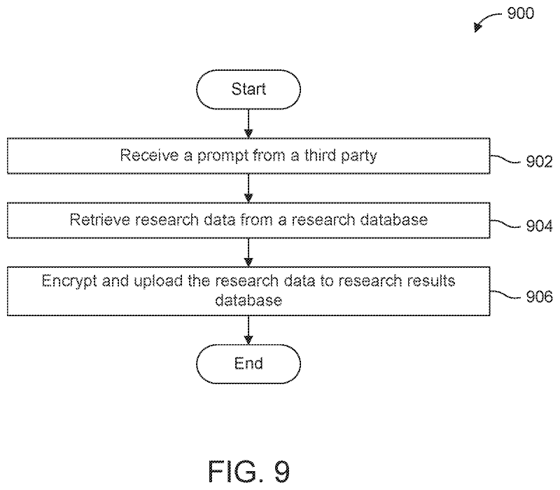

FIG. 9 illustrates a flowchart 900 showing an example method performed by the research upload module 120, according to various embodiments. An example process utilizing the research upload module 120 will now be explained with reference to, for example, the flowchart 900 shown in FIG. 9. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The research upload module 120 may receive a prompt from the third party, at step 902. In various embodiments, the third party may be CROs. The research upload module 120 may retrieve research data from the research database 122, at step 904. In various embodiments, the research data may include data and patients' individual hash (e.g., private keys or public keys) for accessing blockchain. Thereafter, the research upload module 120 may encrypt and upload the research data to the research results database 126, at step 906.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for providing access of a user's health information to third parties, comprising:
providing, from a health information exchange server to a third party, an access to a user's health information based on a user's permission, for performing a research on the user's health information; and
granting the user access, with a user device communicatively coupled with the health information exchange server, a result of the research, wherein the health information exchange server stores health information of a plurality of users over a blockchain network.

2. The computer-implemented method of claim 1, wherein the user's health information comprises blood pressure, heart rate, and number of steps moved per day.

3. The computer-implemented method of claim 1, wherein the third party is an individual belonging to one of hospitals, insurance companies, contract research organizations, and drug companies.

4. The computer-implemented method of claim 1, wherein providing an access to a user's health information comprises receiving an input from the user authorizing an access to a selected portion of the user's health information.

5. The computer-implemented method of claim 1, wherein providing an access to a user's health information comprises receiving a request from the third party to access the user's health information.

6. The computer-implemented method of claim 1, further comprising verifying an accessibility of the user and the third party to the blockchain network.

7. The computer-implemented method of claim 1, further comprising providing to at least one of the user or the third party an encrypted key to access the blockchain network.

8. The computer-implemented method of claim 1, further comprising notifying the user that the user's health information is being used in the research.

9. The computer-implemented method of claim 1, further comprising accessing a third party digital wallet in the blockchain network to update a balance of the third party based on the access to a user's health information.

10. The computer-implemented method of claim 1, further comprising accessing a user digital wallet in the blockchain network to update a balance of the user based on the result of the research.

11. A system for providing access of a user's health information to third parties, comprising:
one or more processors;
a memory coupled to the one or more processors and storing instructions which, when executed by the one or more processors, cause the system to:
provide, from a health information exchange server to a third party, an access to a user's health information based on a user's permission, for performing a research on the user's health information based on an analysis of the user's health information;
allow the user to access, with a user device communicatively coupled with the health information exchange server, a research recommendation is generated to be accessed by the user or the third party, wherein the health information exchange server stores health information of a plurality of users over a blockchain network; and
verify an accessibility of the user and the third party to the blockchain network.

12. The system of claim 11, further comprising a patient data database storing at least a portion of the user's health information including blood pressure, heart rate, and number of steps moved per day.

13. The system of claim 11, wherein the third party is an individual belonging to one of a hospital, an insurance company, a contract research organization, and a pharmaceutical company.

14. The system of claim 11, wherein the one or more processors further execute instructions to provide to at least one of the user or the third party an encrypted key to access the blockchain network.

15. The system of claim 11, wherein the one or more processors further execute instructions to notify the user that the user's health information is being used in the research.

16. The system of claim 11, wherein the one or more processors further execute instructions to access a third party digital wallet in the blockchain network to update a balance of the third party based on the access to a user's health information.

17. The system of claim 11, wherein the one or more processors further execute instructions to access a user digital wallet in the blockchain network to update a balance of the user based on the result of the research.

18. A non-transitory, computer readable medium storing instructions which, when executed by a processor cause a computer to perform a method for providing access of a user's health information to third parties, the method comprising:
providing, from a health information exchange server to a third party, an access to a user's health information based on a user's permission, for performing a research on the user's health information based on an analysis of said user's health information;
granting the user access to, with a user device communicatively coupled with the health information exchange server, a result of the research, wherein the health information exchange server stores health information of a plurality of users over a blockchain network;
receiving an input from the user authorizing an access to a selected portion of the user's health information; and
receiving a request from the third party to access the user's health information.

19. The non-transitory, computer readable medium of claim 18, wherein the method further comprises verifying an accessibility of the user and the third party to the blockchain network.

20. The non-transitory, computer readable medium of claim 18, wherein the method further comprises providing to at least one of the user or the third party an encrypted key to access the blockchain network.
